(19) United States
(12) Patent Application Publication
(10) Pub. No.: US 2017/0136256 A1
(43) Pub. Date: May 18, 2017

(54) QUICK DETACHABLE MAGNETIZED BODY WEAR

(71) Applicant: Bac Ngoc Dinh, Silver Spring, MD (US)

(72) Inventor: Bac Ngoc Dinh, Silver Spring, MD (US)

(21) Appl. No.: 15/347,258

(22) Filed: Nov. 9, 2016

Related U.S. Application Data
(60) Provisional application No. 62/255,727, filed on Nov. 16, 2015.

(51) Int. Cl. A61N 2/06; A44C 15/00; A61N 2/00; A44C 5/00

(52) U.S. Cl. CPC A61N 2/06; A44C 5/0023; A44C 15/0085; A44C 15/005; A61N 2/008; A44D 2203/00

(57) ABSTRACT

A magnetized body wear is configured to be worn on a body part of a user, for example, worn on the wrist or ankle of the feet. The magnetized body wear comprises multiple magnetized spheres coated with a covering material, where the magnetized spheres are removably attached to each other to define a closed loop. One selected pair of the magnetized spheres is detached to open the closed loop and to allow the attached magnetized spheres to be worn and attached around the body part. The closed looped magnetized spheres worn on the body part optimizes bodily conditions of the user.

### CROSS-REFERENCE TO RELATED APPLICATIONS

This application claims priority from the U.S. Provisional Patent Application 62/255,727 filed on Nov. 16, 2015, titled “Comfort coated energizing, stimulating and pain relieving magnetic bracelet or anklet”, which is incorporated herein by reference.

### FIELD OF THE INVENTION

The present invention relates to magnetized devices for human body treatment. More particularly, the present invention relates to a quick detachable magnetized body wear or a magnetized bracelet which is worn on the hands or legs of a user to enhance the physical state of the user.

### BACKGROUND

It is well known the art that there are various magnetized items that are available in the market which are used for ornamental purposes on the human body, such as, magnetized necklaces and bracelets worn on wrist, ankle, or around the neck of the user. Besides the ornamental purpose of such magnetized necklaces and bracelets, they facilitate the flow of the blood based on the magnetic characteristics of the magnetized ornaments. Many of the prior art devices disclose the use of magnetized elements to improvise physical conditions of the human body.

One of such prior arts is the US patent publication 2005/0148809 which discloses a magnetic jewelry which is coated with silver, nickel and black epoxy. However, in this prior art, the coating made of silver is not very effective since the silver oxidizes quickly, and makes the appearance of the magnetic jewelry unattractive. When the magnetic spheres are attached together, they attach to each other in an extremely small surface area. When the magnetic jewelry is moved around, the magnetic force that keeps the magnets in the attachment causes them rub against each other and the outer surfaces of such magnets thereby are easily corroded. This is primarily due to the lack of a protective coating on the outer surface of the magnetic spheres. Further, due to lack of strength of the ceramic magnet, the sphere or any such shape will easily detach from each other, and therefore the wearer may easily lose their magnetic jewelry, which could be a bracelet, anklet, or necklace.

Therefore, there is a need for a magnetized body wear which is configured for optimizing bodily conditions of the user, which can be easily detachable, and which will not corrode during extensive usage.

### SUMMARY OF THE INVENTION

The magnetized body wear disclosed herein addresses the above-mentioned issues. The magnetized body wear is configured to be worn on a body part of a user, for example, neck, wrist or ankle. The magnetized body wear comprises multiple magnetized spheres coated with a covering material, where the magnetized spheres are removably attached to each other to define a closed loop. One selected pair of the magnetized spheres is detached to open the closed loop and to allow the connected magnetized spheres to be worn and attached around the body part. The closed looped magnetized spheres worn on the body part optimizes bodily conditions of the user. In an embodiment, the covering material is configured to prevent corrosion of the magnetized spheres caused by frictional contact between the magnetized spheres during extensive usage.

In an embodiment, the bodily conditions of the user are optimized using magnetized properties of the closed looped magnetized spheres. The closed looped magnetized spheres of the magnetized body wear create a sense of well-being in the body of the user, reduce chronic pain and weakness in the body of the user, and increase energy level in the body of the user. In an embodiment, the size of each magnetized sphere is either of a uniform size or variable size, where the variable size of each magnetized sphere provides a variation in the appearance of the closed looped of magnetized spheres.

In an embodiment, the covering material is a plastic material. In an embodiment, the covering material is one of silicone, acrylic, gold, epoxy, zinc and nickel. In an embodiment, the diameter of the magnetized body wear is configured to be customized to an exact size of the user's body part by increasing or decreasing the number of magnetized spheres, and connecting the magnetized spheres together to define the closed loop of magnetized spheres. In an embodiment, a hole is drilled across each magnetized sphere to introduce a thread member through the drilled hole to hold the magnetized spheres in position. In an embodiment, the magnetized body wear further comprises disc elements inserted between the magnetized spheres to avoid corrosion due to direct contact of the surfaces of the magnetized spheres.

### BRIEF DESCRIPTION OF THE DRAWINGS

FIG. 1 exemplarily illustrates a front perspective view of the magnetized body wear.

FIG. 2 exemplarily illustrates a front perspective view of the magnetized body wear, where one selected pair of the magnetized spheres is detached to open the closed loop.

FIG. 3 exemplarily illustrates a front perspective view of another embodiment of the magnetized body wear, showing a thread member introduced through the magnetized spheres.

FIG. 4 exemplarily illustrates a front perspective view of another embodiment of the magnetized body wear, showing disc elements introduced between adjacent magnetized spheres.

### DETAILED DESCRIPTION OF THE INVENTION

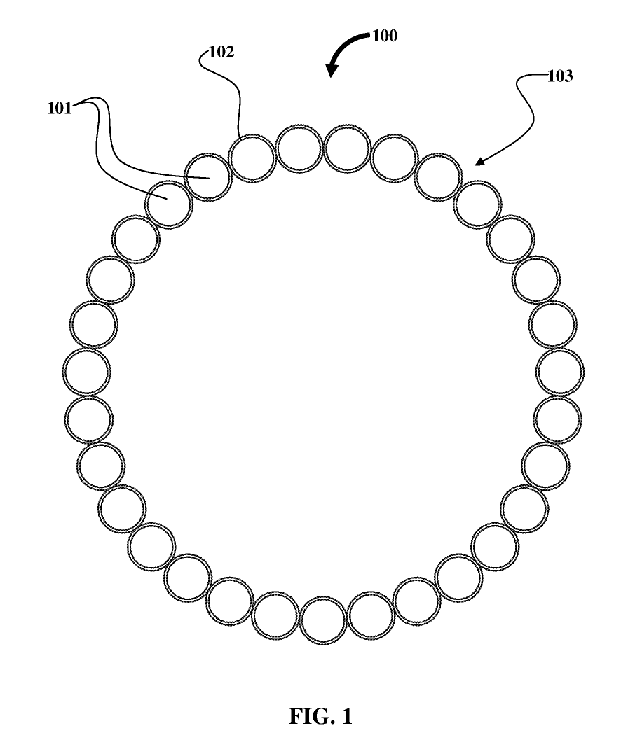

FIG. 1 exemplarily illustrates a front perspective view of the magnetized body wear 100. The magnetized body wear 100 is configured to be worn on a body part of a user, for example, worn on the wrist, ankle or neck. The magnetized body wear 100 comprises multiple magnetized spheres 101 coated with a covering material 102, where the magnetized spheres 101 are removably attached to each other to define a closed loop 103. The magnetized body wear 100 is generally in the form of an anklet or bracelet which combines multiple magnetized spheres 101 which are connected to each other to define the shape of the bracelet or anklet. This bracelet or anklet can be worn on the ankles, wrist, or neck of a user. The magnetized spheres 101 are, for example, neodymium magnets which are comparatively stronger than ceramic magnets used conventionally. When these neodymium magnets are linked together to form the magnetized body wear 100 or the bracelet, anklet, or necklace, the neodymium magnets form a strong bonding to each other which reduces the chances of them detaching from each other or falling off. In an embodiment, the covering material 102 is configured to prevent corrosion of the magnetized spheres 101 caused by frictional contact between the magnetized spheres 101 during extensive usage.

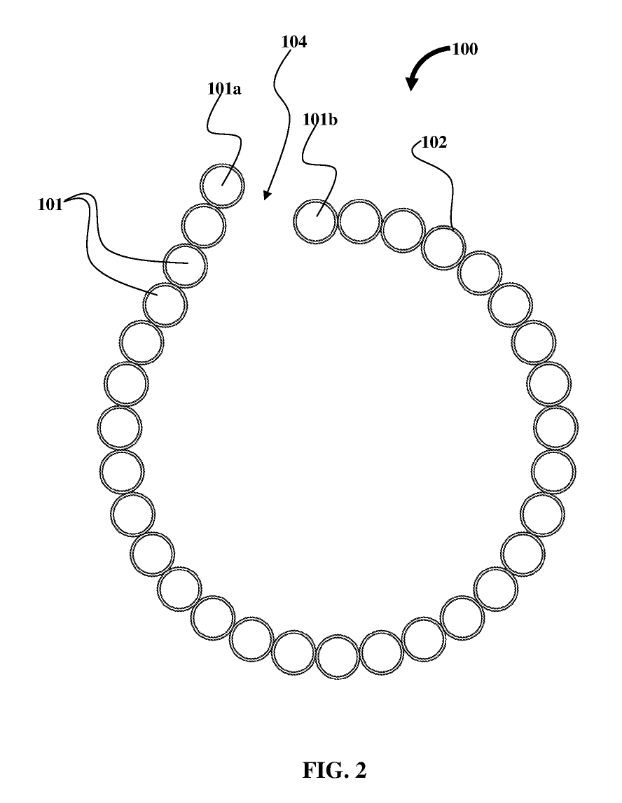

As further described in detail in the FIG. 2, one selected pair 101a and 101b of the magnetized spheres 101 is detached to open the closed loop 103 and to allow the connected magnetized spheres 101 to be worn and attached around the body part. The close looped magnetized spheres 101 worn on the body part optimizes bodily conditions of the user. Due to the magnetic characteristics of the magnetized body wear 100, a magnetic field generated from the magnetized body wear 100 interacts with the body of the user to provide the user with a heightened sense of his or her overall bodily state, which both emotionally and physically enhances the mood and energy level of the user. The magnetized body wear 100 is also referred to as a ‘comfort coated energizing, stimulating and pain relieving magnetic bracelet or anklet’.

In an embodiment, the bodily conditions of the user are optimized using magnetic properties of the closed looped magnetized spheres 101. The closed looped magnetized spheres 101 of the magnetized body wear 100 creates a sense of well-being in the body of the user, reduce chronic pain and weakness in the body of the user, and increase energy level in the body of the user. In general, wearing magnets has been shown to naturally reduce pain from chronic diseases and create a sense of well being, even in those with serious joint problems, pain and muscle aches, and therefore the magnetized body wear 100 utilizes such properties of magnets to improve the health of the users.

In an embodiment, the size of each magnetized sphere 101 is either of uniform size or a variable size, where the variable size of each magnetized sphere 101 provides a variation in the appearance for the closed looped magnetized spheres 101. In an embodiment, the magnetized body wear 100 is constructed with uniformly sized magnetized spheres 101, or with smaller and larger magnetized spheres 101 to vary the appearance of the bracelet or anklet. The magnetized body wear 100 is constructed using varying sizes of magnetized spheres 101 connected to form a circular bracelet fitting the wrist or ankle of the user. The sizes of the magnetized spheres 101 are configured to be varied within a range of, for example, about 0.125 inches, 0.25 inches, 0.375 inches, 0.5 inches, 0.625 inches, and 0.75 inches.

In an embodiment, the covering material 102 is a plastic material. The covering material 102 on the outer layers of the magnetized spheres 101 comprises various types of plastic including silicone and acrylic; or alternatively, gold, epoxy, zinc or nickel. Such materials provide the user with an enhanced usage and unlimited wearing capability. The thin layer of silicone will act as a cushion so when the magnetized spheres 101 are rubbed against each other, it won't easily corrode the metal coating. The thin layer of silicone also prevents metal from oxidization. The extra layer of silicone not only prevents the magnetized spheres 101 from corroding, but also adds comfort for the wearer of the bracelet, necklace or anklet. When the magnetized spheres 101 are coated with silicone or acrylic coating, it generates a magnetized body wear 100 with a variety of colored magnetized spheres 101 available within the color spectrum. The coated covering material 102 adheres to the outside surface of the magnetized spheres 101. The coated magnetized spheres 101 are then connected to each other via the magnetism of each magnetized sphere 101 which is powerful enough to hold the magnetized body wear 100 together.

FIG. 2 exemplarily illustrates a front perspective view of the magnetized body wear 100, where one selected pair 101a and 101b of the magnetized spheres 101 is detached to open the closed loop 103. As shown in FIG. 2, one selected pair 101a and 101b within the linkage of the magnetized spheres 101 is separated to open the closed loop 103, and then the magnetized body wear 100 is manually worn over the ankle, wrist, or neck of the user. In an embodiment, the diameter of the magnetized body wear 100 is configured to be customized to an exact size of the user's body part, such as wrist or ankle, by increasing or decreasing the number of magnetized spheres 101, and connecting the magnetized spheres 101 together to define the closed loop 103 of magnetized spheres 101.

The magnetized body wear 100 is created with the desired color coating to enhance overall comfort and attractiveness. The magnetized body wear 100 is designed to allow the wearer to experience an increased sense of well being and heightened energy levels in the body while reducing joint and body pain via magnetic stimulation. The magnetized body wear 100 is extremely comfortable in terms of ease of wearing and design, and moreover the magnetized body wear 100 is durable, can be worn for any desired period, and can be easily removed. The magnetized body wear 100 is opened by exerting force between two of the magnetized spheres, for example, 101a and 101b, at any point in its circular structure to make an opening 104, whereby the magnetized body wear 100 can be placed on the wrist, or to be easily removed from the wrist. In an embodiment, the magnetized body wear 100 is configured to define a necklace which can be worn around the neck of a user.

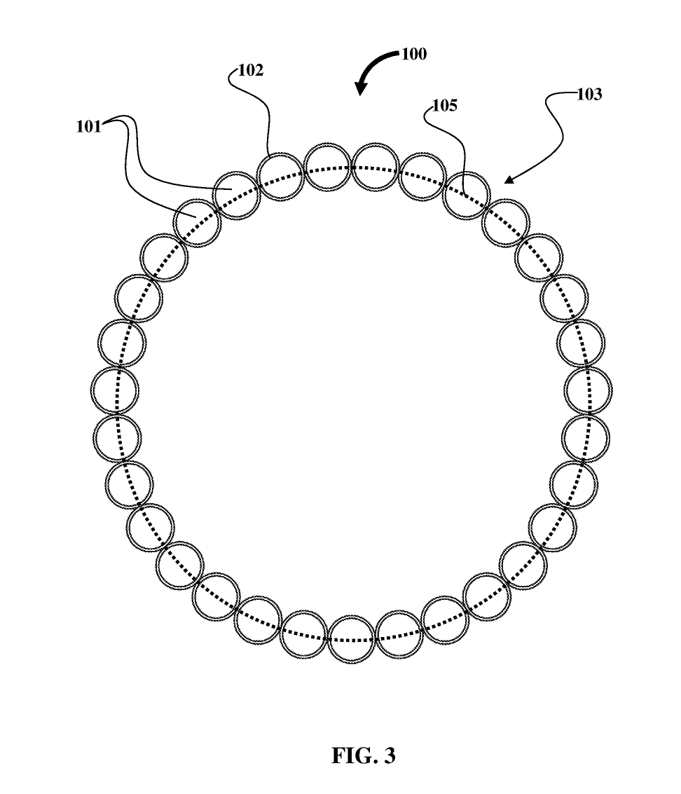

FIG. 3 exemplarily illustrates a front perspective view of another embodiment of the magnetized body wear 100, showing a thread member 105 introduced through the magnetized spheres 101. In an embodiment, a hole is drilled across each magnetized sphere 101 to introduce a thread member 105 through the drilled hole to hold the magnetized spheres 101 in position. The thread member 105 is, for example, includes but not limited to, a non-metallic wire configured to attach the magnetized spheres 101 together which will further prevent the magnetized spheres 101 from falling apart during regular usage. The thread member 105 can be of any material and is not limited to a non-metallic wire. In another example, for a better appearance of the magnetized body wear 100, a hole is drilled in the center of multiple different kind of pearls, sliced semi-precious stones, or Swarovski® crystals, etc., and to insert a magnetized sphere 101 into each of them, and turn them it into the center piece of the bracelet, anklet, or necklace.

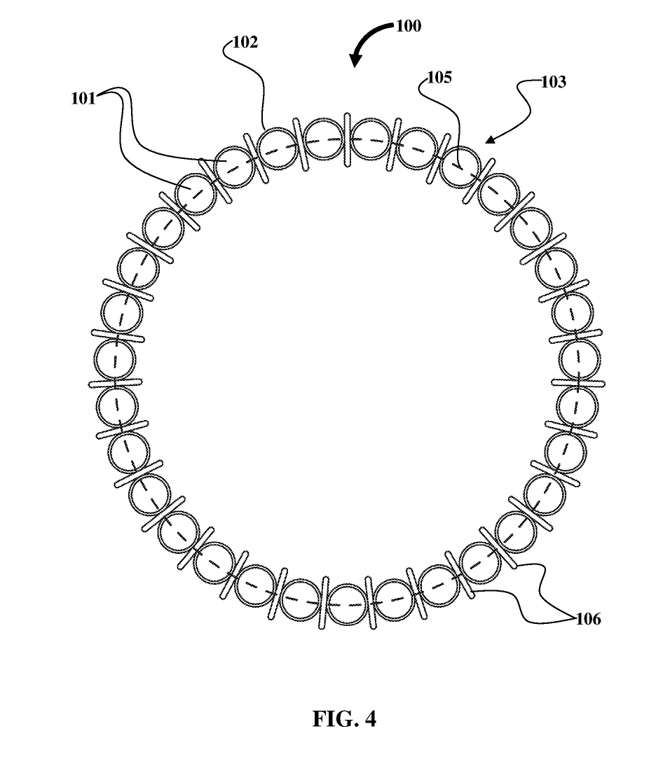

FIG. 4 exemplarily illustrates a front perspective view of another embodiment of the magnetized body wear 100, showing disc elements 106 introduced between adjacent magnetized spheres. To prevent the magnetized body wear 100 from corroding, after the hole is drilled across each magnetized sphere 101 to introduce the thread member 105 as disclosed in FIG. 3, multiple silicone made disc elements 106 are inserted between the magnetized spheres 101 to avoid direct contact of the surfaces of the magnetized spheres 101. The direct contact of the surfaces of the magnetized spheres 101 can induce corrosion due to the rubbing of the adjacent surfaces. In an example, the disc elements 106 can also be substituted by silicon beads. The bead or disc element 106 is, for example, about 1mm (millimeter), 1.5 mm, 2 mm in diameter. In an example, each disc element 106 is made of a different color to increase the aesthetic ornamental appeal of the overall magnetized body wear 100.

The foregoing examples have been provided merely for the purpose of explanation and are in no way to be construed as limiting of the present concept disclosed herein. While the concept has been described with reference to various embodiments, it is understood that the words, which have been used herein, are words of description and illustration, rather than words of limitation. Further, although the concept has been described herein with reference to particular means, materials, and embodiments, the concept is not intended to be limited to the particulars disclosed herein; rather, the concept extends to all functionally equivalent structures, methods and uses, such as are within the scope of the appended claims. Those skilled in the art, having the benefit of the teachings of this specification, may affect numerous modifications thereto and changes may be made without departing from the scope and spirit of the concept in its aspects.

I claim:

1. A magnetized body wear configured to be worn on a body part of a user, the magnetized body wear comprising;
a plurality of magnetized spheres coated with a covering material, wherein the magnetized spheres are removably attached to each other to define a closed loop, wherein one selected pair of the magnetized spheres is detached to open the closed loop and to allow the connected magnetized spheres to be worn and attached around the body part, wherein the closed looped magnetized spheres worn on the body part optimizes bodily conditions of the user.

2. The magnetized body wear of claim 1, wherein the covering material is configured to prevent corrosion of the magnetized spheres caused by frictional contact between the magnetized spheres during extensive usage.

3. The magnetized body wear of claim 1, wherein the bodily conditions of the user are optimized using magnetic properties of the closed looped magnetized spheres, wherein the closed looped magnetized spheres provide to;
create a sense of well being in the body of the user;
reduce chronic pain and weakness in the body of the user; and
increase energy level in the body of the user.

4. The magnetized body wear of claim 1, wherein the size of each magnetized sphere is one of a uniform size and a variable size, wherein the variable size of each magnetized sphere provides a variation in the appearance of the closed looped of magnetized spheres.

5. The magnetized body wear of claim 1, wherein the covering material is a plastic material.

6. The magnetized body wear of claim 1, wherein the covering material is one of silicone, acrylic, gold, epoxy, zinc and nickel.

7. The magnetized body wear of claim 1, wherein a diameter of the magnetized body wear is configured to be customized to an exact size of the user's body part by one of increasing and decreasing number of the magnetized spheres, and connecting the magnetized spheres together to define the closed loop of magnetized spheres.

8. The magnetized body wear of claim 1, wherein a hole is drilled across each magnetized sphere to introduce a thread member through the drilled hole to hold the magnetized spheres in position.

9. The magnetized body wear of claim 8, further comprising disc elements inserted between the magnetized spheres to avoid corrosion due to direct contact of the surfaces of the magnetized spheres.
